(19) United States
(12) Patent Application Publication
(10) Pub. No.: US 2004/0197751 A1
(43) Pub. Date: Oct. 7, 2004

(54) Assessment tool and method for evaluating a person's quality of life

(76) Inventor: Albert A. Alexander, Coventry, CT (US)

Correspondence Address: MCCORMICK, PAULDING & HUBER LLP, CITY PLACE II, 185 ASYLUM STREET, HARTFORD, CT, 06103, US

(21) Appl. No.: 10/816,565

(22) Filed: Apr. 1, 2004

Related U.S. Application Data
(60) Provisional application No. 60/460,204, filed on Apr. 3, 2003.

(51) Int. Cl. A61B 5/02; G09B 19/00

(52) U.S. Cl. 434/236

(57) ABSTRACT

The present invention provides an assessment tool and method for evaluating a person's quality of life based on a plurality of personal attributes. The assessment tool includes a list of a plurality of personal attributes determined to be significant to a person's quality of life and means for establishing a score for each of the personal attributes for a selected person. A display representing the established score for each of the personal attributes is also provided, wherein the display provides a visual representation of the selected person's quality of life with respect to the personal attributes.

### CROSS REFERENCE TO RELATED APPLICATIONS

This application claims priority to U.S. Provisional Patent Application No. 60/460,204 filed on Apr. 3, 2003 and titled “Personal Stock Index”. The disclosure of the provisional application is incorporated in its entirety herein by reference.

### FIELD OF THE INVENTION

The present invention generally relates to personal assessment methods and apparatus and in particular to an assessment tool and method for evaluating a person's quality of life with respect to certain personal attributes.

### BACKGROUND OF THE INVENTION

Most currently used analysis tools including assessment and self-assessment tools are directed to a specific area of a person's life such as stress levels, sleep habits, eating habits, drinking habits, anger management, conflict resolution, and other similar or related topics. None of these tools measure or evaluate the overall quality of life of a person. Further, none of the above-identified prior art assessment tools provide a tool for evaluating a person's overall quality of life with respect to personal qualities or personal attributes that are determined to be relevant to a person's quality of life. Additionally, none of the above-identified prior art analysis or self-assessment tools include a display providing a visual representation of the results of the analysis.

Based on the foregoing, it is the general object of the present invention to provide an assessment tool and method for evaluating a person's quality of life that improves upon, or overcomes the problems and drawbacks associated with prior art assessment tools.

### SUMMARY OF THE INVENTION

The present invention is directed to an assessment tool for evaluating a person's quality of life based on a plurality of personal attributes. The present invention assessment tool includes a list identifying a plurality of personal attributes determined to be significant to a person's quality of life. The assessment tool also provides means for establishing a score for each of the personal attributes for a selected person being evaluated. A display is provided for recording the established score for each of the personal attributes evaluated and provides a visual representation of the selected person's quality of life with respect to the evaluated personal attributes.

In one embodiment of the present invention assessment tool, the list of personal attributes determined to be relevant to a person's quality of life includes at least one of the following: 1) belief in God, 2) health, 3) personal hygiene, 4) family relationships, 5) education, 6) socio-economic status, 7) political status, 8) patriotic status, 9) interracial relationships or tolerance, 10) ethics, 11) morality, 12) social status, 13) personal relationships, and 14) self-defense skills. The above-identified personal attributes are included in one embodiment of the assessment tool designed to evaluate the quality of a person's life. Depending on the person or group of persons on which the present invention assessment tool is being utilized, the above-identified list of personal attributes can be modified depending on the personal characteristics determined to be relevant to a person's quality of life to the person or group of persons being evaluated. Alternatively, the personal attributes determined to be relevant to a person's quality of life can be identified by the person or persons performing the evaluation. In the case of a self-evaluation process, the person performing the evaluation and being evaluated are the same.

The means for establishing a score for each of the personal attributes identified as relevant to the quality of life for a selected person can include a list of questions related to each of the personal attributes identified. The questions can include questions designed to provoke thought and reflection as to the selected person's characteristics, actions or activities related to the particular personal attribute. Alternatively, the questions can be directed to specific issues wherein a specific answer is used in determining a score for the personal attribute being evaluated.

If the assessment tool is to be used as a self-assessment tool, the means for establishing a score for each of the personal attributes can include soul-searching and self-evaluation processes including an examination of personal thoughts and feelings relative to each of the personal attributes being assessed. Further, a review of one's personality or personal motivations related to each of the personal attributes can be included. Also, a self-evaluation process can include reflection of one's own thoughts, beliefs and behavior as to various issues involved with the person's life and circumstance related to each of the personal attributes being evaluated. Additionally, in the case of an evaluation of a person's health or personal hygiene, a self-evaluation process can include an examination of one's physical body or parts thereof.

In a preferred embodiment, the means for establishing a score for each of the personal attributes includes determining a score in a range from 0 to 10 for each of the personal attributes. The range of scores can be adjusted to be a larger range or smaller range depending on the personal attributes being assessed or the means for establishing a score for each of the personal attributes. If the assessment tool is being used as a self-assessment tool, the person being evaluated establishes a score for each of his/her own personal attributes determined to be relevant to the person's quality of life. In the preferred embodiment of the present invention assessment tool, a score for each of the personal attributes is established following a review of a plurality of questions related to each of the personal attributes. Alternatively, the score for each of the personal attributes can be established following a review of the person's character, actions or activities related to each of the personal attributes without reviewing any particular questions.

The display provides a means of recording and displaying the results of an assessment of one's quality of life with respect to the personal attributes reviewed. The display includes an identifier or label for each of the personal attributes and means to record and display the score established for each of the personal attributes. Accordingly, the display provides a visual representation of a person's quality of life with respect to the personal attributes reviewed.

The present invention further includes means for designating the established score with a first designation if the established score is equal to or below a predetermined minimum score and a second designation if the established score is above the minimum score. In other embodiments, three or more designations can be used to distinguish a person's score in each of the personal attributes.

The display provides can be used as a reminder for further self-analysis or as a motivational tool for improving one's life in those areas related to personal attributes either designated with the first designation or otherwise having an established score that is less than a desired score. Those personal attributes designated with the second designation and displayed as such can be used as a reminder to a person of a desire for continued or further personal achievements in those areas of his/her life.

In another embodiment of the present invention assessment tool and method, the assessment tool is replicates the stock market wherein each of the personal attributes are identified and referred to as personal stocks and the total or composite score of all of the personal stocks combined represents a personal stock index. Additionally, in the stock market embodiment of the present invention assessment tool, a personal stock having an established score equal to or below a predetermined minimum score is designated as a bear market and a personal stock having an established score above the minimum score is designated as a bull market.

### BRIEF DESCRIPTION OF THE DRAWINGS

FIG. 1 is a perspective view of a display according to the present invention.

FIG. 2 is partial view of another embodiment of a display according to the present invention including a bar graph.

FIG. 3 is partial view of another embodiment of a display according to the present invention including a vertical graph formed of pegs positioned on a board defining holes.

FIG. 4 is partial view of another embodiment of a display according to the present invention including a horizontal graph formed of pegs and positioned on a board defining holes.

FIG. 5 is partial view of a computer-enabled embodiment of the present invention.

### DETAILED DESCRIPTION OF THE INVENTION

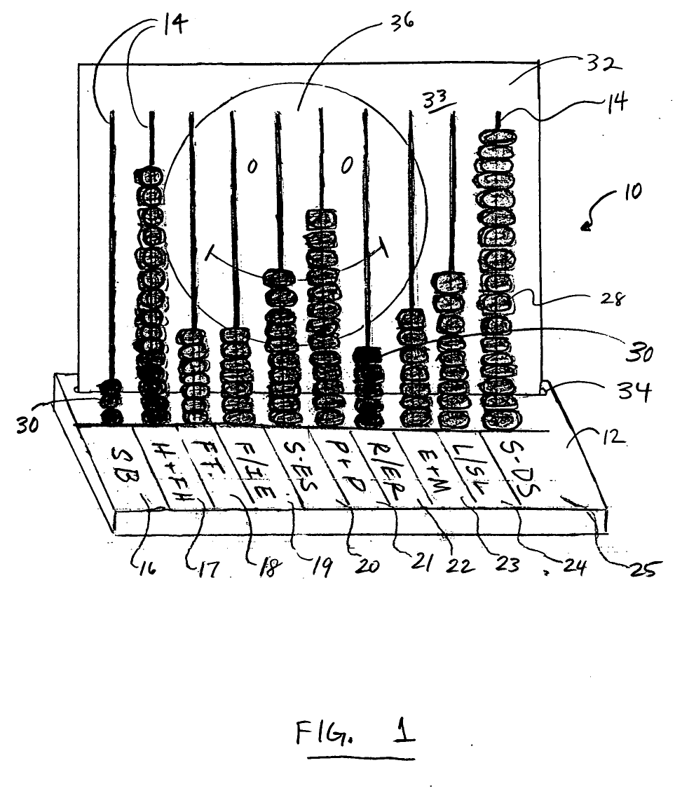

FIG. 1 shows one embodiment of a display according to the present invention generally designated by the reference number 10. The display 10 includes a base 12 and a plurality of rods 14 attached to the base. Each of the rods 14 includes an associated label 16-25 identifying a certain personal attribute of a selected person. The labels 16-25 are interchangeable such that the order of the personal attributes can be changed on the display 10. Additionally, the labels 16-25 can be revised depending on the personal attributes selected to be represented on the display 10.

In the FIG. 1 embodiment, the display 10 includes rods 14 for each of the following personal attributes identified with the reference numerals 16-25: 16) SB—Supreme Being; 17) H+FH—Health, Fitness and Personal Hygiene; 18) FT—Family Ties; 19) F/I E—Formal/Informal Education; 20) S-ES—Social-Economic Status; 21) P+P—Politics and Patriotism; 22) R/ER—Race/Ethnic Relations; 23) E+M—Ethics and Morality; 24) L/SL—Love/Social Life; and 25) S-DS—Self Defense Skills. The above-identified personal attributes 16-25 are included in a preferred embodiment of the assessment tool and method of the present invention. The personal attributes evaluated and displayed on the display 10 can be revised depending on the desired use of the assessment tool and the person or type of persons being evaluated.

Still referring to FIG. 1, the display 10 includes a plurality of beads 28 defining holes therein stacked on the rods 14. Each stack of beads 28 positioned on the rods 14 represent a score for the corresponding personal attribute identified by the labels 16-25 associated with the rod 14. In one embodiment of the present invention, a score between 0 and 10 is established for each of the personal attributes identified by the labels 16-25. A method for establishing the scores is discussed infra. Beads 30 of a first color are used to designate a score that is below a predetermined minimum score such as 5 and beads 28 of a second color are used to designate a score equal to or above the minimum score. Alternatively, a bead 30 of the first color could be used to designate a score equal to the minimum score and a bead 28 of the second color could be used to designate a score greater than the minimum score.

The colored beads 28 and 30 are used in the display 10 as visual indicators representative of either a satisfactory level (above the minimum score), or an unsatisfactory level (below the minimum score) of a person's performance or proficiency in each of the personal attributes displayed. The minimum score in the preferred embodiment is designated as five; however, the minimum score can be adjusted by a user depending on personal preference as to a minimum acceptable score. Additionally, the minimum score can also be adjusted by a user depending on the personal attributes being assessed. For example, a user might determine that for his purposes, a score of eight should be the minimum score in the Health and Personal Hygiene 17 category. Accordingly, if his score for the attribute of Health and Personal Hygiene 17 is established at a score equal to 7, then the beads 30 of the first color would be used on a rod 14 corresponding to the Health and Personal Hygiene category 17 indicating a performance level less than the minimum score.

The display 10 includes a background member 32 removably disposed in an opening 34 defined by the base 12. The background member 32 in the illustrated embodiment includes a smiley face 36 depicted on a surface 33 of the background member. The smiley face 36 is symbolic of a generally favorable score in the personal attributes labeled 16-25. The background member 32 is interchangeable with various other background members 32 provided that include different symbols on a surface 33 thereof. For example, the background member can include a smiley face 36 with a large smile, or a small smile. Alternatively, the smiley face 36 on the surface 33 can be replaced with a poker face, a frown, a face with tears, or a crying face. Additionally, other background features could be included on the surface 33 of the background member 32 such as neutral or relaxing scenes. In the FIG. 1 embodiment, the background member 32 is reversible wherein the background member 32 can be removed from the opening 34 rotated approximately 180 degrees and re-inserted in the opening 34 such that the smiley face 36 on the surface 33 thereof can be replaced with another illustration as discussed above. In other embodiments of the display 10, the features on the surface 33 of the background member 32 have no significance or correlation to the displayed score represented by the beads 28 and 29.

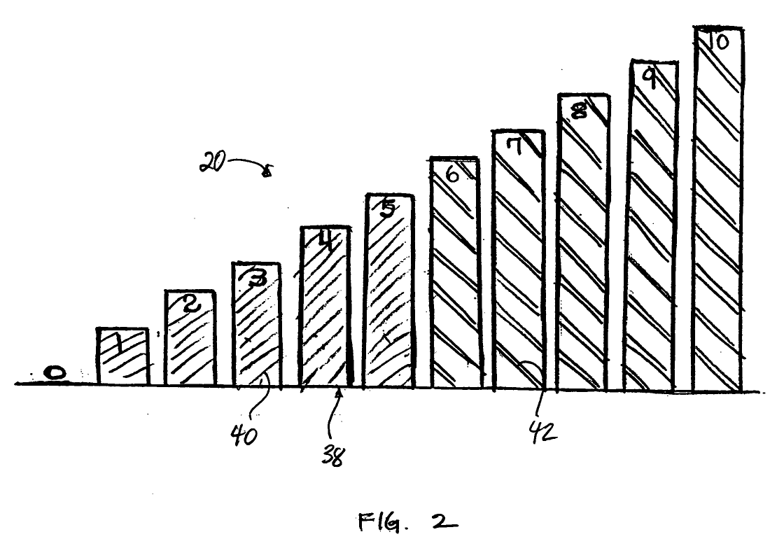

FIG. 2 is a partial view of a display 20 according to another embodiment of the present invention wherein a score established for each personal attribute is represented with a bar 38 such that the display 20 forms a bar graph. As shown in FIG. 2, the bars 38 having heights 1-5 corresponding to a score below the minimum score are of a first color 40 and the bars having heights 6 and over corresponding to scores above the minimum score are of a second color 42.

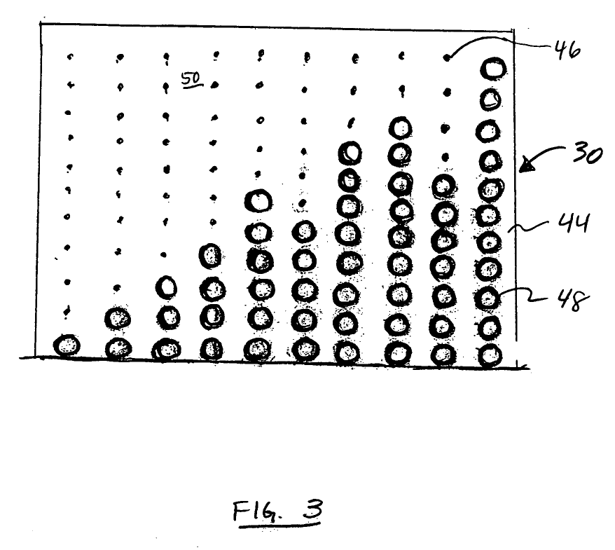

FIG. 3 is a partial view of a display 30 according to another embodiment of the present invention. The display 30 includes a board 44 that defines a plurality of holes 46 for receiving pegs 48. The score for each personal attribute is represented with one or more pegs 48 inserted in the holes 46. The pegs 48 can include pegs having first and second colors as discussed above with respect to the beads 28 and 29. The board 44 can include a smiley face 36 or other illustrations on a front surface 50 thereof as discussed above with respect to the FIG. 1 embodiment.

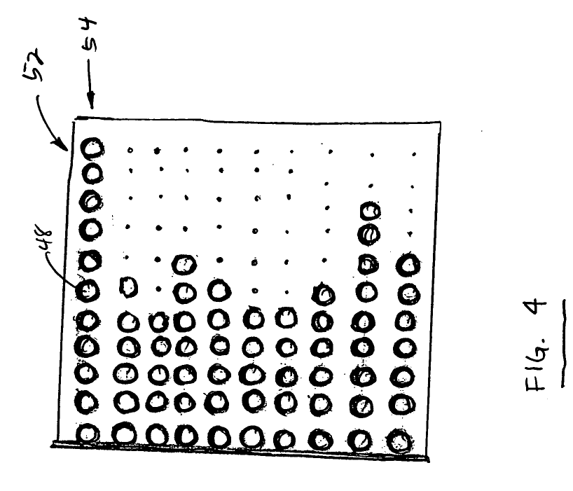

FIG. 4 is a partial view of a display 52 according to the present invention wherein the pegs 48 are inserted in horizontal rows 54 wherein each horizontal row represents the score for one of the personal attributes assessed.

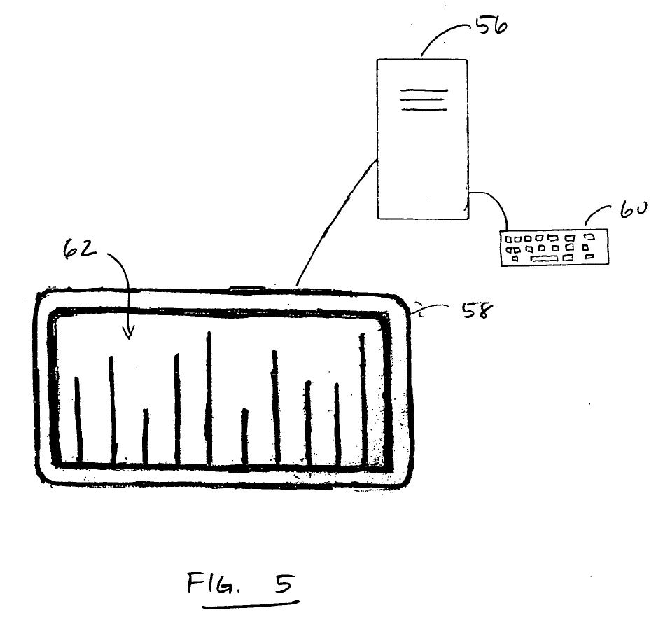

FIG. 5 shows an embodiment of the present invention adapted to operate on a computer 56 which includes a monitor 58 and an input device 60. The computer 56 includes a software application stored thereon embodying the assessment tool of the present invention. As shown in FIG. 5, an image of a display 62 represents a person's score for each of the personal attributes identified with the labels 16-25 (not shown).

In one embodiment of the present invention, a score for each of the personal attributes 16-25 is established in part by considering a plurality of questions related to each of the personal attributes. The person performing the assessment can review a plurality of questions related to the personal attribute being considered and reflect on the character or attitude of the person being assessed in view of each question. The questions may not have one correct answer and can be used only for the purpose of provoking thought or contemplation of the life of a person being assessed or certain portions thereof. The questions provided are not required to establish a score for each personal attribute. Thus, a score for each personal attribute can be established in other ways without departing from the scope of the invention. Additionally, the particular questions for each of the personal attributes can be revised or customized depending on the circumstances of the particular person or group of persons for which the assessment tool is being used.

Following is a list of questions developed by the Applicant for use in a preferred embodiment of the present invention wherein the assessment tool is a self-assessment tool. The questions listed are related to the corresponding personal attributes identified with the labels 16-25 and a range of the total score to be established for each of the personal attributes.

16. SUPREME BEING, (Score: integer in the range of 0 to 10)

A. Do you believe in GOD (monotheism) who is creator and ruler of the and known by revelation (theist)?

1. Do you practice (what you preach) the tenets of your religion or are you:

a) pretense of virtue, piety, etc.?

b) would you rather hear a sermon, see a sermon or be a sermon?

B. Do you believe in the doctrine that GOD is not a personality, but that all laws, forces, manifestation, etc. of the self-existing Universe are God (pantheism)?

C. Do you believe in or worship many GODS (polytheism)?

D. Do you believe in GOD as a creative, moving force, but otherwise reject formal religion and its doctrines of revelation, divine authority, etc. (deist)?

E. Do you question the existence of GOD, heaven, etc, in the absence of material proof and in unwillingness to accept supernatural revelation (agnostic)?

F. Do you believe that there is no God and reject all religious belief and deny the existence of GOD (atheist)?

17. HEALTH FITNESS and PERSONAL HYGIENE, (Score: integer in the range of 0 to 10)

A. How do you rate your physical, mental and emotional health?

B. Are you conscious of your total fitness?

C. Which do you think is currently more dominant in your life:

1. Genetic code/gifts

2. Chronological age

3. Physiological age

4. Mental age

5. Psychological age, etc.?

D. Do you see a doctor for regular check-ups, eat a balanced diet, get sufficient and restful sleep, exercise properly, inhale and exhale properly, recreate regularly, evince a smut of humor and engage in genuine laughter.

E. Are you satisfied with your personal hygiene (health maintenance)?

18. FAMILY TIES, (Score: integer in the range of 0 to 10)

A. Is there requited love and respect for most members of your immediate and/or extended family?

B. Do you experience a sense of serenity and security in the company of your family?

C. Do you sense that members of your immediate/extended family reciprocate accordingly?

D. Do you attempt to reconcile differences with your family regardless of point(s) of contention?

E. Are you compassionately aware of the sentiments and needs of your immediate and/or extended family without joining with them in expressing negative ideas?

F. Do you believe that spirituality, requited veracity, love and happiness are the primary forces that bind families together?

19. FORMAL/INFORMAL EDUCATION, (Score: integer in the range of 0 to 10)

A. Are you satisfied with your level of formal/informal education?

B. Do you continue to expand your formal/informal educational horizons (maximize your brain power)?

C. Do you feel comfortable engaging in cerebral activities?

D. Are you satisfied with your critical thinking skills, analytical skills, etc.?

E. Are you computer literate?

20. SOCIO-ECONOMIC STATUS, (Score: integer in the range of 0 to 10)

A. Are you satisfied with your current socio-economic status (based on your financial goals, objectives, time lines, etc)?

B. Are you solvent (have you factored in inflation)?

C. Do you have anti-prosperous or anti-risk attitudes and behaviors?

D. Are you satisfied with your self-worth and net-worth?

E. Have you diversified your investment portfolio (regardless of the magnitude)?

F. Are you familiar with basic business principles (including insurance principles)?

21. POLITICS and PATRIOTISM, (Score: integer in the range of 0 to 10)

A. Do you vote (frequently, infrequently)?

B. Are you satisfied with the executive, legislative and judicial branches of government? Do you trust your government?

C. Are you pleased with domestic and foreign affairs? Are you active in government?

D. Do you feel that your representatives represent your needs? Do you feel disenfranchised?

E. Do you pledge your allegiance to your country, while zealously attempting to cause it to live up to it's constitution (for all people)?

22. RACE/ETHNIC RELATIONS, (Score: integer in the range of 0 to 10)

A. Do you believe that the concept of human rights go to the very core of relationships among all people?

B. Do you attempt to translate the concepts of human rights into realities expressed in attitude, behavior/practice?

C. Do you believe that national origin, racial identity, religious preference, economic status etc., should not add to or detract from the worth, dignity or equality of opportunity of an individual?

D. Do you encourage and actively participate in fostering cultural diversity and equality in all facets of society?

E. Do you need to subordinate a person or group in order to falsely enhance your self-concept/worth?

23. ETHICS and MORALITY, (Score: integer in the range of 0 to 10)

A. Are you satisfied with your standards of conduct and moral judgement?

B. Are you satisfied with your code of ethics?

C. Do you conform to the rules of propriety?

D. Are you comfortable with your moral quality, character and image?

E. Are you concerned about the ethics and morality of your friends/peers?

24. LOVE/SOCIAL LIFE, (Score: integer in the range of 0 to 10)

A. Do you have an amorous or romantic relationship?

B. Is your affection requited/unrequited?

C. Do you set a positive tone of harmony, compassion and goodwill for time spent with people?

D. In an effort to establish or maintain friendships, do you match the negative energy of others without realizing it?

E. Do you value connecting and empathizing with people of all races, ethnicities, and cultures?

F. Is it easier for you to negate the virtues of a person than to match them?

G. Are the qualities you enjoy in a friend the qualities you express as a friend?

H. Have you met your soul mate/kindred spirit?

25. SELF-DEFENSE SKILLS, (Score: integer in the range of 0 to 10)

A. Are you satisfied with your self-defense skills?

B. Have you taken the necessary precautions to defend your house and home?

C. Do you feel secure in a large gathering or unfamiliar places?

D. Do you avoid contentious people and exercise self-restraint?

E. Will you dial 911 or run, run, run?

Once a score is established for each of the personal attributes, the display 10 can be arranged to record and display the established score for each of the personal attributes 16-25. The display provides a visual representation of a person's quality of life with respect to the personal attributes 16-25. For satisfactory scores or those above the minimum score, the display 10 can be used as a reminder to a person to maintain their current levels of achievement or scores with respect to each of the personal attributes displayed. In the event the established scores identify achievements in one or more of the personal attributes that are below the minimum score or indicate a performance below a desired level of achievement, the display can be periodically reviewed and used as a motivational tool to promote further growth and achievements in those areas. If the present invention assessment tool including the display 10 is incorporated into a person's daily life and periodically used, the assessment tool can promote personal growth with respect to those personal attributes identified to be important to the person's quality of life. Accordingly, the assessment tool of the present invention can be used to improve a person's overall quality of life.

The present invention also provides a method of evaluating a person's quality of life based on a plurality of personal attributes. The method includes determining a plurality of personal attributes significant to a person's quality of life, establishing a score for each of the personal attributes for a selected person and displaying the established score for each of the personal attributes for the selected person. The display providing a visual representation of the selected person's quality of life with respect to the personal attributes selected.

The foregoing description of embodiments of the invention has been presented for the purpose of illustration and description, it is not intended to be exhaustive or to limit the invention to the form disclosed. Obvious modifications and variations are possible in light of the above disclosure. The embodiments described were chosen to best illustrate the principals of the invention and practical applications thereof to enable one of ordinary skill in the art to utilize the invention in various embodiments and with various modifications as suited to the particular use contemplated. It is intended that the scope of the invention be defined by the claims appended hereto.

What is claimed is:

1. An assessment tool for evaluating a person's quality of life based on a plurality of personal attributes, the assessment tool comprising:
a list of a plurality of personal attributes determined to be significant to a person's quality of life;
means for establishing a score for each of the personal attributes for a selected person;
a display representing the established score for each of the personal attributes; and wherein
the display provides a visual representation of the selected person's quality of life with respect to the personal attributes.

2. The assessment tool according to claim 1 further comprising means for designating the established score with a first designation if the established score is equal to or below a predetermined minimum score and a second designation if the established score is above the minimum score.

3. The assessment tool according to claim 1 wherein the list of personal attributes includes at least one of a person's belief in God, health, personal hygiene, family relationships, education, socio-economic status, political status, patriotic status, interracial relationships or tolerance, ethics, morality, social status, personal relationships, and self-defense skills.

4. The assessment tool according to claim 1 wherein the assessment tool is a self-assessment tool including wherein a user determines at least one of the plurality of personal attributes or establishes a score for each of the personal attributes.

5. The assessment tool according to claim 1 wherein the means for establishing a score for each of the personal attributes includes. a list of one or more questions related to each of the personal attributes.

6. The assessment tool according to claim 1 wherein the display includes a bar graph.

7. The assessment tool according to claim 1 wherein the display includes an abacus.

8. The assessment tool according to claim 1 further comprising a computer including a memory, a user interface, a display device and software stored on the memory, wherein the assessment tool is operable on the computer.

9. The assessment tool according to claim 1 wherein the established score for each of the personal attributes is in a range between about 0 and about 10.

10. The assessment tool according to claim 2 wherein the first and second designations include first and second colors.

11. A method of evaluating a person's quality of life based on a plurality of personal attributes, the method comprising the steps of:
determining a plurality of personal attributes significant to a person's quality of life;
establishing a score for each of the personal attributes for a selected person;
displaying the established score for each of the personal attributes for the selected person; and wherein
the method providing a visual representation of the selected person's quality of life with respect to the personal attributes.

12. The method of claim 11 further comprising a step of designating each displayed score with a first designation if the score is equal to or below a predetermined minimum score and a second designation if the score is equal to or above the minimum score.

13. The method of claim 11 wherein the step of determining a plurality of attributes includes selecting at least one of, belief in God, health, personal hygiene, family relationships, education, socio-economic status, political status, patriotic status, interracial relationships or tolerance, ethics, morality, social status, personal relationships, and self-defense skills.

14. The method of claim 11 wherein the step of establishing a score for each of the personal attributes for a selected person includes reviewing at least one question related to each of the personal attributes of the selected person.
